(19) United States
(12) Patent Application Publication
(10) Pub. No.: US 2005/0003371 A1
(43) Pub. Date: Jan. 6, 2005

(54) Modified nucleotides and methods of labeling nucleic acids

(75) Inventors: Jack D. Anderson, Oceanside, CA (US); Jeffrey Carl Braman, Carlsbad, CA (US); Haoqiang Huang, Milwaukee, WI (US)

Correspondence Address: PALMER & DODGE, LLP;KATHLEEN M. WILLIAMS / STR, 111 HUNTINGTON AVENUE, BOSTON, MA, 02199, US

(73) Assignee: Stratagene

(21) Appl. No.: 10/691,269

(22) Filed: Oct. 22, 2003

Related U.S. Application Data
(60) Provisional application No. 60/420,675, filed on Oct. 23, 2002.

(51) Int. Cl. C12Q 1/68; C07H 21/04

(52) U.S. Cl. 435/6; 536/25.32

(57) ABSTRACT

The invention relates to functionalized nucleotides that permit the covalent linkage of the nucleotides to moieties containing reactive groups complementary to the functional group. More specifically, the invention relates to nucleotides comprising a functional group attached to the nucleobase that permits the covalent attachment of the nucleotides or a nucleic acid comprising such nucleotides to detectable moieties, polypeptides and solid supports. The invention further relates to nucleic acids comprising such functionalized nucleotides, methods of labeling nucleic acids with such nucleotides, and kits comprising such nucleotides.

This application claims the priority of U.S. Provisional Patent Application No. 60/420,675, filed Oct. 23, 2002, the entirety of which is incorporated herein, including figures.

### BACKGROUND OF THE INVENTION

Detectably labeled nucleotides and nucleic acids are widely used in research and diagnostic methods. Fluorescently labeled nucleotides and nucleic acids have gained favor as an alternative to radiolabeled compositions, due to factors such as stability, cost, safety and disposal issues. Fluorescently-labeled nucleotides and nucleic acids are commonly used in techniques such as DNA sequencing, in-situ hybridization and PCR-based research and diagnostic methods(see, e.g., Ansorge, et al., 1987; Prober, et al., 1987; Connell, et al., 1987; Lichter, et al., 1991; Selleri, et al., 1991). Fluorescent dNTPs can be used for internal labeling of nascent DNA chains (Schubert, et al., 1990; Voss, et al., 1991; Voss, et al., 1992). Not only are the fluorescent properties of the fluorescent dyes required for various procedures using fluorometric detection techniques, but the dye may also act as a hapten for an anti-dye (e.g., anti-fluorescein) antibody-alkaline phosphatase conjugate.

Fluorescently modified nucleotides can also be used to generate labeled primers and thus provide a quick, low-cost alternative to methods utilizing machine synthesis. Several groups have reported that terminal deoxynucleotidyl transferase (TdT) incorporates a single fluorescein or biotin-UTP analog at the 3′ terminus of oligonucleotides (Kossel and Roychoudhury, 1971; Dirks, et al., 1991; Kumar, et al., 1988). These modified oligos were shown by others to function identically to 5′-end labeled oligos when used for PCR and fluorescence-based DNA sequencing (Igloi and Schiefermayr, 1993). These results demonstrate the use of fluorescent nucleotides in an economical, one-step oligonucleotide labeling procedure.

Other available systems for nonradioactive detection of nucleic acids rely, for example, on the formation of a stable interaction between probe and reporter enzyme-conjugate. These systems utilize, for example, biotin-, fluorescein-, or digoxigenin-labeled probes which are incubated during the detection procedure with streptavidin-, fluorescein antibody- or digoxigenin antibody-reporter enzyme conjugates, respectively. The biotin-avidin and hapten-antibody systems have the potential to produce acceptable levels of sensitivity but are subject to certain limitations. The production of nonspecific signal (background) inherent with the use of streptavidin-reporter enzyme conjugates in membrane hybridization assays has been attributed to nonspecific adsorption of streptavidin to the hybridization membrane. Lability of the biotin-streptavidin interaction in a solid phase transcription assay has also been reported (Fujita and Silver, 1993). Accordingly, there exists a need for an improved method of attaching reporter enzymes to nucleic acids. An improved method should exhibit strong (e.g., covalent) conjugation of the nucleic acid to the enzyme and exhibit low non-specific binding.

The use of amino nucleotides has been reported for TdT-catalyzed labeling of oligonucleotides similar to the procedure described above for the fluorescent nucleotides (Reyes and Cockerell; Lightsmith II, Promega, Madison Wis.). Precedent is found in the literature for the successful use of DNA probes made by chemical crosslinking of a reporter enzyme with amine-labeled oligonucleotides. Probes were made by chemical conjugation of an oligonucleotide containing an amine-modified base to a reporter enzyme using the homobifunctional crosslinking reagent disuccinimidyl suberate (DSS). Such probes made possible the nonisotopic detection of plasmids immobilized onto membranes at sensitivity levels ranging from 1×10⁻¹⁷ to 1×10⁻¹⁹ moles depending upon the enzyme/substrate system chosen (Jablonski, et al., 1986; Ruth, 1994). This compared favorably to the sensitivity achieved using ³²P, which was detected at a level of 1×10⁻¹⁹ moles.

Many bioconjugates have been made using heterobifunctional crosslinking reagents that contain a maleimide (maleimidylcyclohexane carboxylate, MCC) functionality (Means and Feeney, 1990; Partis, et al.,1983; Bernatowicz and Matsueda, 1986). This group exhibits high reactivity towards thiols.

U.S. Pat. No. 5,516,641 describes the reaction of nucleotides comprising a sulfhydryl on the sugar moiety with a maleimide on a contiguous nucleotide.

U.S. Pat. No. 4,749,647 describes a ribonucleoside, 5-aminouridine triphosphate, derivatized such that the primary amine at the 5-position is coupled to produce a nucleotide containing a reactive maleimide group.

Nampalli et al. describe standard chain terminator dideoxynucleotides comprising maleimide, pyridyl dithio and bromoacetyl groups for linkage to a label (Nampalli et al., 2002, Bioconjugate Chem. 13: 468-473).

### SUMMARY OF THE INVENTION

The invention provides nucleotides bearing functional groups that simplify the process of covalently joining detectable groups or solid supports to the nucleotides and nucleic acids comprising them. The invention also provides methods of labeling nucleic acids with such nucleotides, as well as kits containing such nucleotides.

The invention encompasses a nucleotide comprising the structure:
Phosphate-Sugar-Nucleobase-Linker-F;
   wherein F is a functional group selected from:

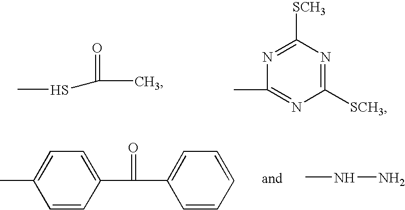

The invention also encompasses a nucleic acid (including an oligonucleotide or polynucleotide) comprising such a nucleotide.

In one embodiment, the linker is attached to the nucleobase at the N-4 or C-5 position of the nucleobase when the nucleobase is a pyrimidine, or at the N-6, C-8 or C(N)-7 position of the nucleobase when the nucleobase is a purine.

In another embodiment, the nucleobase is selected from the group consisting of: adenine, cytosine, guanine, thymine, uracil and hypoxanthine.

In another embodiment, the linker is selected from the group consisting of:
—CH₂—(CH₂—CH₂)ᵥ—CH₂—NHC(O)-Q-; —CH₂—(CH₂—CH₂)ᵥ—CH₂—C(O)—NH—C(O)-Q-;
—S—CH₂C(O)-Q-; —S—CH₂CH₂NH—C(O)-Q-; —O—CH₂C(O)-Q; —O—CH₂CH₂NH—C(O)-Q-;
—NH—(CH₂)ᵥ—NH—C(O)-Q-; 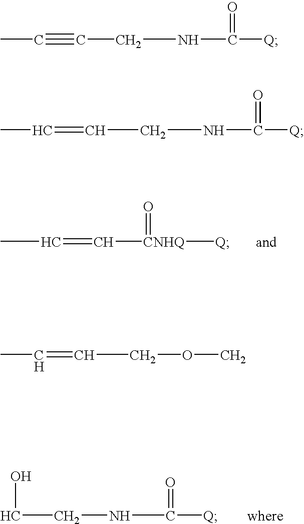
v=0,1,2,3, Q=—NH(CH₂)₆NH—, —NH—(CH₂)₂—NH, —(CH₂)₅NH—, —(CH₂)₂—C(O)—NH—(CH₂)₃—O—(CH₂)₂—O—(CH₂)₂—O—(CH₂)₃—NH—, —NH—[(CH₂)₂—O—)_{W}—(CH₂)₂—NH—, —(CH₂)₂ C(O)—NH—[(CH₂)₂—O]_{W}—NH—, and w=2,3,4,5.

In another embodiment, the nucleotide is selected from the group consisting of ATP, dATP, ddATP, GTP, dGTP, ddGTP, CTP, dCTP, ddCTP, UTP, dUTP, dTTP and ddTTP.

In another embodiment, the phosphate moiety is a mono-, di-, tri-, or tetraphosphate group.

In another embodiment, the sugar moiety is a cyclic pyranofuranose sugar. In a more specific embodiment, the cyclic pyranofuranose sugar is selected from the group consisting of ribofuranosyl, 2′-deoxyribofuranosyl, and 2′,3′-dideoxyribofuranosyl.

In another embodiment, the sugar moiety is a cyclic non-furanose sugar. In a more specific embodiment, the cyclic non-furanose sugar is selected from the group consisting of oxetan, pyran or oxadiazepine.

In another embodiment, the sugar moiety is an acyclic sugar analog. In a more specific embodiment, the acyclic sugar analog is selected from the group consisting of phosphonomethoxyethyl, 2-oxyethoxymethyl, 2-hydroxymethoxymethyl, and 3-pentenyl.

The invention further encompasses a method of labeling a nucleotide comprising the structure: Phosphate-Sugar-Nucleobase-Linker-F, wherein F is as described above, the method comprising contacting the nucleotide with a detectable moiety comprising a reactive thiol group.

In one embodiment, the detectable moiety comprises a chromogenic dye, a fluorescent dye, a polypeptide or an enzyme.

The invention further encompasses a nucleic acid comprising such a labeled nucleotide.

The invention further encompasses a method of labeling a nucleic acid, the method comprising contacting the nucleic acid with a nucleotide comprising the structure: Phosphate-Sugar-Nucleobase-Linker-F, wherein F is a functional group as described above. In one embodiment, the contacting is performed in the presence of a nucleic acid polymerase. The invention further encompasses a nucleic acid labeled in this manner.

In one embodiment, the method further comprises contacting the nucleotide with a thiol-containing detectable moiety.

In one embodiment, the thiol-containing detectable moiety is a chromogenic moiety, a fluorescent dye, a polypeptide or an enzyme.

The invention further encompasses a method of attaching a nucleic acid to a solid support, the method comprising: a) contacting the nucleic acid with a nucleotide comprising the structure: Phosphate-Sugar-Nucleobase-Linker-F, wherein F is as described above, in the presence of a nucleic acid polymerase, wherein the contacting results in the incorporation of the nucleotide into the nucleic acid or its complement; b) contacting the nucleic acid of step (a) with a solid support comprising a reactive group complementary to the functional group F on the nucleotide, wherein the contacting results in covalent attachment of the nucleic acid of step (a) to the solid support.

In one embodiment, the solid support is a plate, tube, bead or column matrix.

The invention further encompasses a kit comprising a nucleotide comprising the structure: Phosphate-Sugar-Nucleobase-Linker-F; wherein F is a functional group as described above. In one embodiment, the kit further comprises a nucleic acid polymerase, and packaging materials therefor.

The invention further encompasses a nucleotide comprising the structure:
Phosphate-Sugar-Nucleobase-F
   wherein F is a functional group selected from: 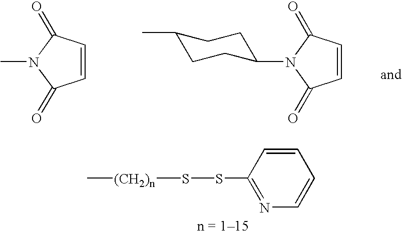
   wherein Sugar is an acyclic sugar analog.

In one embodiment, the acyclic sugar analog is selected from the group consisting of phosphonomethoxyethyl, 2-oxyethoxymethyl, 2-hydroxymethoxymethyl, and 3-pentenyl. The invention further encompasses a polynucleotide comprising such a nucleotide, and a kit comprising such a nucleotide. In one embodiment the kit further comprises a nucleic acid polymerase and packaging materials therefor.

The term “nucleotide” as used herein refers to a phosphate ester of a nucleoside, e.g., mono, di, tri, and tetraphosphate esters, wherein the most common site of esterification is the hydroxyl group attached to the C-5 position of the pentose (or equivalent position of a non-pentose “sugar moiety”).

As used herein, the term “phosphate moiety” refers to a mono-, di-, tri- or tetraphosphate. A phosphate moiety as used herein can comprise one or more substitutions, including substitutions of sulfur for one or more oxygen atoms.

As used herein, “sugar moiety” refers to a moiety which occupies a position in the nucleotide relative to the other components of the nucleotide which is equivalent to the position occupied by the pyrofuranose sugar ring in a traditional nucleotide (i.e., ATP, dATP, CTP, dCTP, etc). A “sugar moiety” as used herein may be a pyrofuranose sugar ring comprising a hydroxyl group at both the 2′ and 3′ carbons, or wherein one or both of the hydroxyl groups bonded to the 2′ and 3′ carbons is replaced with —H. A “sugar moiety” as used herein also refers to a non-pyrofuranose sugar ring including, but not limited to the following cyclic structures: 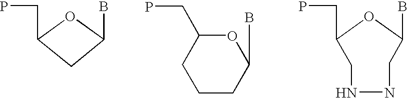
wherein B is a nucleobase linked to a fluorescent moiety, and wherein P is a polyphosphate moiety. A nucleotide can bear a sugar moiety differing from the pyrofuranose sugar ring of a traditional nucleotide, but as used herein, the nucleotide bearing an alternative sugar moiety must be capable of recognition and incorporation by a nucleic acid polymerase. Alternatively, a “sugar moiety” as used herein may refer to an acyclic group which occupies the same position in the nucleotide as the pyrofuranose sugar ring in a traditional nucleotide, provided that the nucleotide analog comprising the acyclic sugar moiety is capable of being enzymatically incorporated into a polynucleotide chain in a manner similar to that of a nucleotide which contains a pyrofuranose sugar ring. Such acyclic moieties include, but are not limited to the following structures: 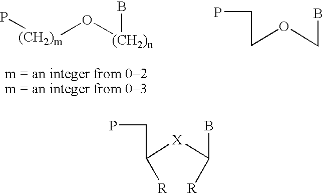
wherein B is a nucleobase, P is a polyphosphate moiety, X is CH₂ or CF, and R is CH₃, CH, or CF. A nucleotide bearing an alternative group in place of the standard sugar moiety will often be incorporated and/or terminate polymerization with greater or lesser efficiency than the standard nucleotides; where desired, polymerase enzymes can be tailored according to methods known in the art in order to improve the incorporation/termination efficiency with respect to a given alternative nucleotide structure.

As used herein, the term “nucleobase” refers to the heterocyclic nitrogenous base of a nucleotide or nucleotide analog. Nucleobases useful according to the invention include, but are not limited to adenine, cytosine, guanine, thymine, uracil, and hypoxanthine. Additional nucleobases that can be comprised by a nucleotide according to the invention include, but are not limited to naturally-occurring and synthetic derivatives of the preceding group, for example, pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine. Nucleobases useful according to the invention will permit a nucleotide bearing that nucleobase to be enzymatically incorporated into a polynucleotide chain and will form Watson-Crick base pairs with a nucleobase on an antiparallel nucleic acid strand.

As used herein, the phrase “Watson-Crick base pair” refers to a pair of hydrogen-bonded nucleobases on opposite antiparallel strands of nucleic acid. The well-known rules of base pairing first elaborated by Watson and Crick, require that adenine (A) pairs with thymine (T) or uracil (U), and guanine (G) pairs with cytosine (C), with the complementary strands anti-parallel to one another. The Watson-Crick pairing rules can be understood chemically in terms of the arrangement of hydrogen bonding groups on the heterocyclic bases of the oligonucleotide, groups that can either be hydrogen bond donors or acceptors. In the standard Watson-Crick geometry, a large purine base pairs with a small pyrimidine base; thus, the AT base pair is the same size as a GC base pair. This means that the rungs of the DNA ladder, formed from either AT or GC base pairs, all have the same length. Further recognition between bases is determined by hydrogen bonds between the bases. Hydrogen bond donors are heteroatoms (nitrogen or oxygen in the natural bases) bearing a hydrogen; hydrogen bond acceptors are heteroatoms (nitrogen or oxygen in the natural bases) with a lone pair of electrons. In the geometry of the standard Watson-Crick base pair, a six membered ring (in natural oligonucleotides, a pyrimidine) is juxtaposed to a ring system composed of a fused six membered ring and a five membered ring (in natural oligonucleotides, a purine), with a middle hydrogen bond linking two ring atoms, and hydrogen bonds on either side joining functional groups appended to each of the rings, with donor groups paired with acceptor groups.

As used herein, the term “Watson-Crick base pair” encompasses not only the standard AT, AU or GC base pairs, but also base pairs formed between nucleobases of nucleotide analogs comprising non-standard or modified nucleobases, wherein the arrangement of hydrogen bond donors and hydrogen bond acceptors permits hydrogen bonding between a non-standard nucleobase and a standard nucleobase or between two complementary non-standard nucleobase structures. One example of such non-standard Watson-Crick base pairing is the base pairing engaged in by the nucleotide analog inosine, wherein the hypoxanthine nucleobase forms two hydrogen bonds with adenine, cytosine or uracil.

As used herein, the term “linker” refers to the chemical group or groups that join a functional group, as the term is defined herein, to the nucleobase on a nucleotide according to the invention.

As used herein, the term “functional group” refers to a chemical group that is reactive with a complementary reactive group to form a covalent bond between the molecule comprising the functional group and that comprising the complementary reactive group. A functional group according to the invention can exist in a protected form, such that it is not immediately reactive with a complementary group, but will be reactive upon deprotection. Examples of functional groups useful according to the invention include, but are not limited to thioacetyl (complementary reactive groups include, for example, maleimide and iodoacetate), di-S-methyl triazine (complementary reactive groups include nucleophiles, for example, amines, hydroxyls and thiols), a benzoylbenzoic group (complementary reactive groups are nucleophilic groups), and a hydrazino group. Additional functional groups include, for example, maleimide, pyridine dithioalkyl and bromoacetyl groups.

As used herein, the term “reactive thiol group” refers to a thiol group (SH) that is not protected from reaction, e.g., by disulfide (S—S) bond formation. A reactive thiol group results, for example, from the reduction of a disulfide bond.

As used herein, “detectable moiety” refers to a moiety that can be directly or indirectly detected. Detectable moieties include, but are not limited to radionuclides (e.g., ³²P, ³³P, ³⁵S, etc.), chromophores, fluorophores, fluorescence quenchers, enzymes, enzyme substrates, affinity tags (e.g., biotin, avidin, streptavidin, etc.), and epitope tags recognized by an antibody. As used herein, a “directly detectable” moiety can be measured without requirement for additional substrates or binding partners. Examples of directly detectable moieties include radionuclides and fluorophores. As used herein, an “indirectly detectable” label requires reaction or interaction with another substrate or reagent for detection. Examples of indirectly detectable labels include enzymes (requires substrate), enzyme substrates (requires enzyme), affinity tags (requires affinity partner), and epitope tags (requires antibody).

As used herein, the phrase “nucleic acid polymerase” refers an enzyme that catalyzes the template-dependent polymerization of nucleoside triphosphates to form primer extension products that are complementary to one of the nucleic acid strands of the template nucleic acid sequence. A nucleic acid polymerase enzyme initiates synthesis at the 3′ end of an annealed primer and proceeds in the direction toward the 5′ end of the template. Numerous nucleic acid polymerases are known in the art and commercially available. One group of preferred nucleic acid polymerases are thermostable, i.e., they retain function after being subjected to temperatures sufficient to denature annealed strands of complementary nucleic acids.

As used herein, the term “terminal transferase” or “terminal deoxynucleotidyl transferase” refers to an enzyme that catalyzes the addition of at least one deoxyribonucleotide to the terminal 3′-hydroxyl of a DNA strand. Terminal transferase enzymes are widely available commercially.

As used herein, the term “solid support” refers to a solid or semi-solid (e.g., a gel matrix) material to which a nucleotide according to the invention or a nucleic acid comprising such a nucleotide is attached. Solid supports include, but are not limited to functionalized glass, membranes, charged paper, nylon, cellulose, germanium, silicon, PTFE, polystyrene, gallium arsenide, agarose, agar, acrylamide, tresyl and epoxy resins, gold and silver. Any other material known in the art that is capable of having functional groups such as maleimide, amino, carboxyl, thiol or hydroxyl incorporated on its surface is contemplated. The format of the support can be, for example, plates (e.g., tissue culture or microtiter plates), tubes (e.g., polystyrene tubes), beads or microbeads, or column matrices (e.g., agarose, Sephacryl (Pharmacia, Uppsala, Sweden), Sephadex (Pharmacia), Sepharose (Pharmacia), etc.). Suitable solid supports are available commercially, and will be apparent to the skilled person.

### BRIEF DESCRIPTION OF THE FIGURES

FIG. 1 shows the structures of several exemplary functionalized nucleotides according to the invention.

FIG. 2 schematically shows several representative variations on linkers and points of attachment for the linker arm on nucleobase moieties.

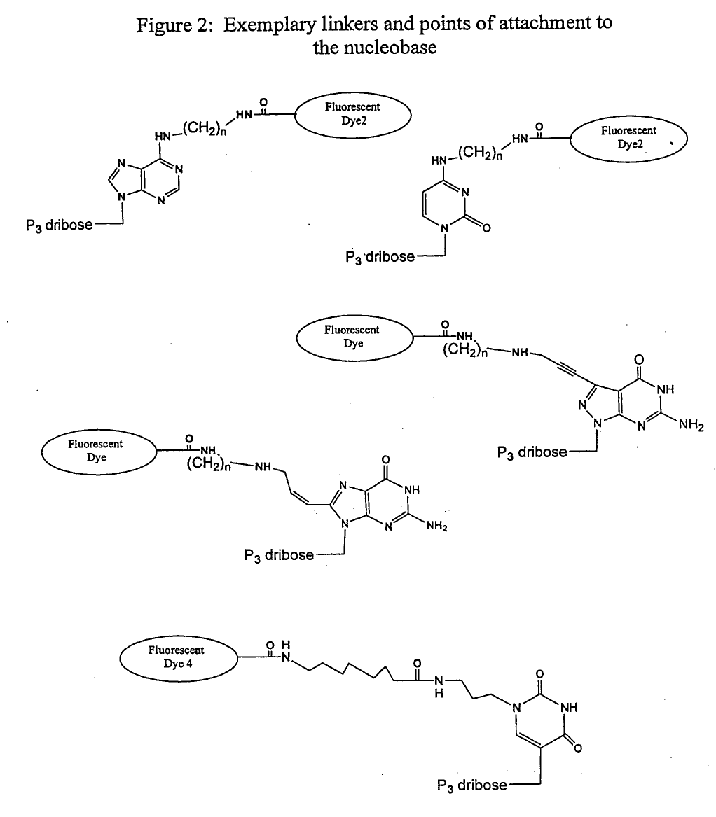

FIG. 3 shows autoradiograms of reactions using PDP-dUTP (pyridine dithiopropanoate-dUTP) and various DNA polymerases. (A) 3′-end labeling of a 12-mer oligonucleotide using PDP-dUTP and terminal transferase according to Procedure 1: Lane 1, reaction in the absence of dUTP (dTTP) analog; lane 2, reaction including PDP-dUTP. (B) primer extension reaction using PDP-dUTP and T7 DNA polymerase following Procedure 2: Lane 1, kinased 12-mer only; lane 2, reaction in the presence of PDP-dUTP; lane 3, reaction in the absence of any dUTP (dTTP) analog. (C) incorporation results using PDP-dUTP and Taq DNA polymerase following Procedure 2: Lane 1, reaction in the presence of PDP-dUTP; lane 2, reaction in the absence of any dUTP (dTTP) analog. Top arrows indicate the position of a ³²P labeled 18-mer oligonucleotide marker. Bottom arrows indicate the position of a 12-mer oligonucleotide marker. The incorporation of nucleotides labeled using any of the functional groups described herein can be monitored in a similar fashion.

FIG. 4 shows autoradiograms of primer extension reactions using SAc-dUTP and various DNA polymerases. (A) Incorporation results using SAc-dUTP and the Klenow fragment of DNA polymerase I following Procedure 3: Lanes 1 and 2, primer extension reactions in the presence of SAc-dUTP, isolated fractions 1 and 2, respectively; Lane 3, reaction in the absence of dUTP (dTTP) analog, top arrow indicates the position of a ³²P labeled 63-mer oligonucleotide marker, bottom arrow indicates the position of a 17-mer oligonucleotide marker. (B) Incorporation results using SAc-dUTP and Taq DNA polymerase following Procedure 3: Lanes 1 and 2, primer extension reactions in the presence of SAc-dUTP, isolated fractions 1 and 2, respectively; Lane 3, reaction in the absence of dUTP (dTTP) analog. (C) 3′-end labeling reaction using SAc-dUTP and TdT following Procedure 1: Lanes 1 and 2, reactions in the presence of SAc-dUTP, fractions 1 and 2, respectively; lane 3, reaction in the absence of dUTP (dTTP) analog. The incorporation of nucleotides labeled using any of the functional groups described herein can be monitored in a similar fashion.

FIG. 5 shows a forty minute exposure of dot blots containing serial dilutions of PCR amplified IL 2 gene hybridized against alkaline phosphatase-tailed oligonucleotide probes made using the modified nucleotide, MCC-dUTP (maleimido-methylcyclohexane-dUTP). “A” and “B” refer to probes made with 2.7 nmole and 5.4 nmole, respectively, of MCC-dUTP in the labeling reaction. Nucleic acids labeled through use of any of the functional groups described herein can be used in a similar manner.

FIG. 6 shows chemiluminescent signal resulting from dot blots containing dilutions of two different genes hybridized against alkaline phosphatase-tailed oligonucleotide probes made using the modified nucleotide, PDP-dUTP, according to Procedure 5 (see text). Columns labeled CHAP, 1 and 2 contain PCR amplified human IL2 gene spotted at 1/10 serial dilutions, rows 1-3, and PCR amplified actin gene (row 4) as a negative control. Columns 3-5 contain PCR amplified actin gene spotted at 1/10 serial dilutions, rows 1-3, and PCR amplified IL2 (row 4) as a negative control. Lanes 1 and 2 refer to variable amounts of starting anti-IL2 oligonucleotide included in the end labeling reaction (10 μg and 32 μg, respectively). CHAP refers to a control hybridization using 0.5 μL of an alkaline phosphatase probe made from a machine-synthesized, thiol-tailed oligonucleotide. Lanes 3-5 refer to variable amounts of starting anti-actin oligonucleotide which were tailed with PDP-dUTP (16, 3.8 and 10 μg, respectively). Nucleic acids labeled through use of any of the functional groups described herein can be used in a similar manner.

FIG. 7 shows chemiluminescent signal resulting from hybridization of a Southern transfer of single and multiple copy genes with oligonucleotide-alkaline phosphatase conjugates made using PDP-dUTP according to Procedure 5. (A) lanes 1 and 2: Southern hybridization of 5 μg and 1 μg aliquots, respectively, of gel-fractionated human genomic DNA with a probe homologous to the human IL2 gene (band seen at the site indicated by the arrow). (B) lanes 1 and 2: Southern hybridization of 5 μg and 1 μg aliquots, respectively, of gel-fractionated human genomic DNA detected with a probe homologous to the actin gene. Nucleic acids labeled through use of any of the functional groups described herein can be used in a similar manner.

FIG. 8 shows chemiluminescent detection of fluorescein-labeled riboprobes on (A) Southern and (B) Northern blots using a still video imaging system (Procedure 9). “M” indicates lanes loaded with fluorescein-labeled Lambda Hind III markers. (A) Lanes 1-5 were loaded with 10 μg of human genomic DNA mixed with 500 μg, 100 μg, 10 μg, 1 pg and 0.5 μg, respectively, of target pBluescript® DNA. (B) Detection of human alpha 1-antitrypsin gene in a Northern transfer of mouse total and messenger RNA. Lanes 1-3 contain 2 μg of mouse messenger RNA, lanes 4 and 5 contain 10 μg and 20 μg of mouse total RNA, respectively. Riboprobes made with modified nucleotides (e.g., ribonucleotides or analogs recognized by an RNA polymerase) labeled by reaction with functional groups described herein can be used in a similar manner.

FIG. 9 schematically shows steps in the synthesis of di-S-methyl triazine (bis-methylthio-1,3,5-triazine) and its attachment to a nucleotide (dUTP).

FIG. 10 schematically shows steps for the activation and coupling of methylthio-triazinyl-dTUP to a label.

FIG. 11 schematically shows the structure of a nucleotide bearing the hydrazino functional group.

FIG. 12 shows the structures of the maleimidyl, maleimido-methylcyclohexane and pyridine-dithioalkyl functional groups used in evaluating the labeling approaches described herein.

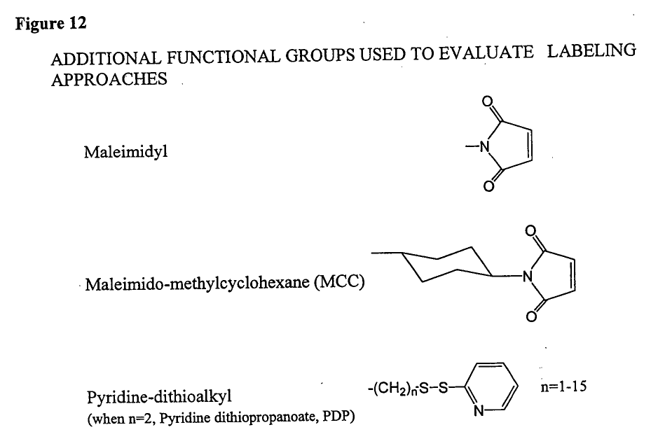

### DETAILED DESCRIPTION OF THE INVENTION

The invention provides nucleotide analogs with functionalities permitting the attachment of moieties comprising a complementary reactive group. Nucleotide analogs according to the invention can contain functionalities appropriate for homo-bifunctional crosslinking reactions, as well as for heterobifunctional crosslinking reactions. Modified nucleotides according to the invention can be used in a variety of procedures, including, for example, attachment of dyes, polypeptides (e.g., transcription factors or other polypeptides), enzymes (e.g., detectable enzymes, such as luciferase, P-galactosidase, etc.), antibodies, epitope tags or other specific binding reagents to a nucleic acid comtaining the functionalized nucleotide, or for the attachment of functionalized nucleic acids to solid supports comprising a complementary reactive group.

Modified Nucleotides According to the Invention:

In one aspect, the invention provides nucleotide analogs bearing functional groups that permit the covalent attachment of the nucleotide analogs or nucleic acids comprising them to moieties comprising complementary reactive groups. The invention provides nucleotides of the general structure:
Phosphate-Sugar-Nucleobase-Linker-F
   wherein F is a functional group that permits the coupling of the modified nucleotide to an entity comprising a reactive group complementary to the functional group. The functional group F useful according to the invention includes an S-acetyl group, a di-S-methyl triazine group, a benzoylbenzoic group a hydrazino group and functionally equivalent variations of these. Structures of these functional groups are shown below.

### Examples of Functional Groups Useful According to the Invention

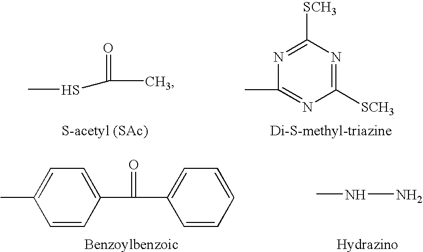

The thioacetyl group (SAc) is reactive with maleimide groups as well as with iodoacetate groups present on, for example, detectable moieties (e.g., fluorescent dyes or proteins) or solid supports. DNA labeled with the masked-thiol nucleotide, S-Ac-dUTP must be deprotected (deacetylated) first in order to express active thiol functionalities suitable for coupling to thiol-reactive labels/proteins. This is accomplished following the protocol for deacetylation described in the product literature for acetylated thiol-products (i.e., SATA product # 26102, from Pierce Chemical). A suitable method for deacetylation includes admixture of a deacetylation solution containing hydroxylamine. Further details are provided in Example 8, below.

Di-S-methyl triazine (or more accurately, bis-methylthio-1,3,5,-triazine) is also useful as a functional group according to the invention. After an oxidation step, di-S-methyl triazine is reactive with any nucleophile, e.g., an amine, thiol, hydroxyls, etc. located on the entity to be attached to the modified nucleotide. The di-S-methyl-triazine label is unreactive in its initial state on the triazine-dUTP nucleotide but can be activated for displacement by nucleophiles (e.g., amines) after enzymatic incorporation of the modified nucleotide into the DNA. This is done by exposure of the di-S-methyl triazine to an oxidizing source. In the presence of oxidants like persulfate, perborate, etc. the sulfur residues on the triazine are oxidized to the sulfoxide/sulfone state thereby activating the adjacent ring carbons for nucleophilic attack.

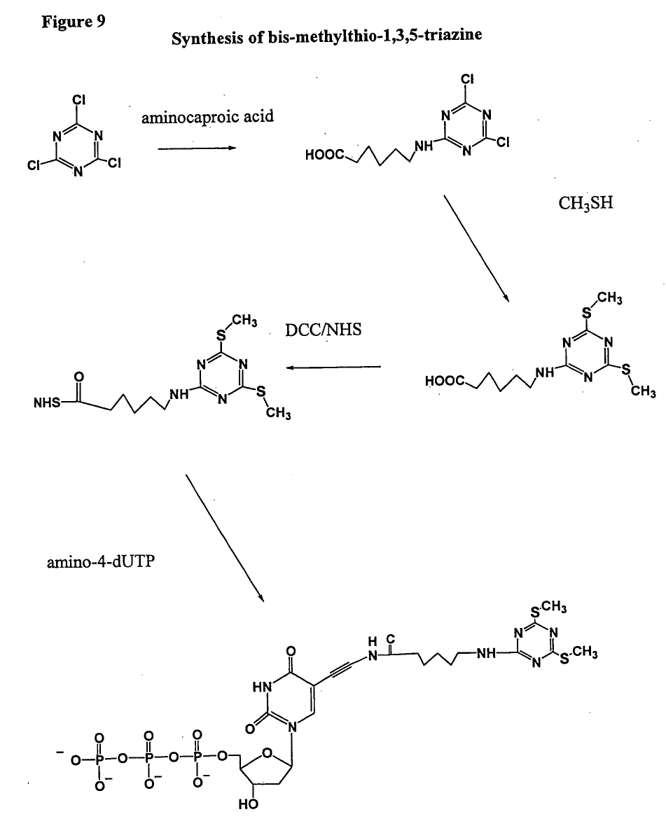

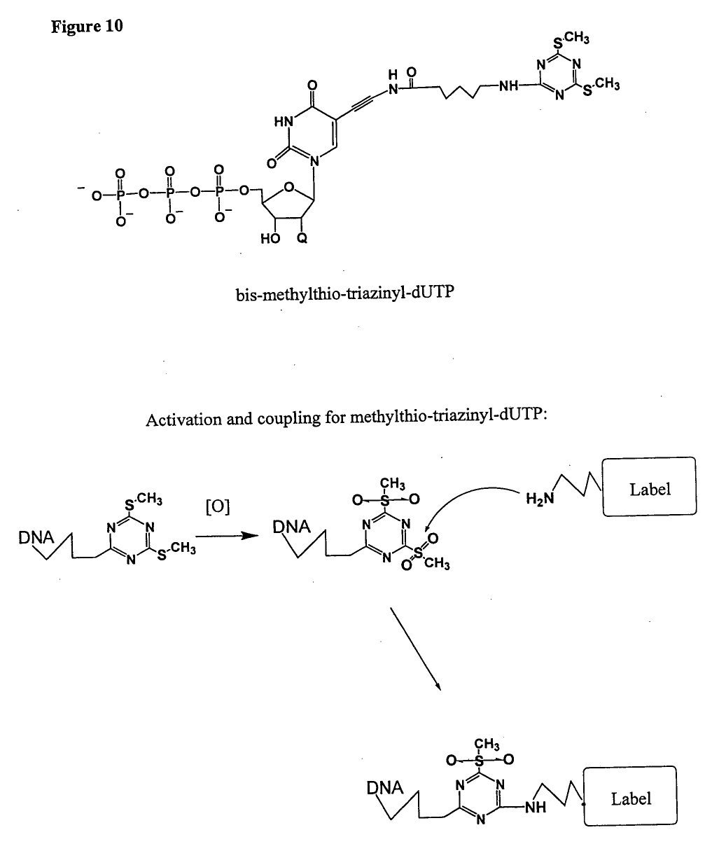

The synthetic steps for the generation of di-S-methyl triazine labeled nucleotide begin with cyanuric chloride, as depicted in the schematic diagram of FIG. 9. The resultant bis-sulfonyltriazine becomes reactive for nuclophilic displacement/attachment similar to adducts of cyanuric chloride (trichloro-1,3,5-triazine) which has often been used as a functional group for facilitating protein/nucleic acid labeling. To have a label initially unreactive is a favorable property—this ensures that the label will react only at the desired time point—suppressing unwanted side reactions which preliminarily deactivate a portion of the label and thereby reduce coupling yields. The activation and coupling for a nucleic acid modified with, for example, methyl thio-triazinyl-dUTP, is shown in FIG. 10.

Conditions for oxidizing the di-S-methyl-triazine are similar to periodate oxidation of RNA. For example: dissolve 100-300 pmole of di-S-methyl-triazine-dUTP-labeled nucleic acid (DNA, RNA, etc.) in 60 uL of water containing 0.5 mg of NaIO₄. Incubate 1-2 hrs in the dark at room temperature. Add 20 uL of 10% ethylene glycol to stop the reaction, incubating 10 min. Reaction products are then ethanol precipitated by adding 600 uL of water, and sodium acetate (pH 5.2) to 0.25 M followed by 2.5 volumes of EtOH. Precipitated products are then centrifuged and desalted with 100 uL of 70% EtOH.

Conditions for using the di-S-methyl-triazine functionality to label nucleic acid are similar to using adducts of cyanuric chloride. Following oxidation of the labeled nucleic acid, amino-modified label in 0.1 M acetate, pH 5 is admixed with the precipitated methylsulfonyl-labeled-DNA and the reaction is heated (if required) to 60 degrees for 1 hr.

The benzolybenzoic functionality, e.g., on benzoylbenzoic-dUTP, is another example of a functional group useful according to the invention. Benzophenones (the benzolybenzoic group) are photo-activatable. Photo-activation is accomplished by, e.g., exposure to long-wave UV light. Photoreactive crosslinking reagents are important tools for determining the proximity of two sites. Thus, these probes can be employed to define relationships between two reactive groups on a protein, on a ligand and its receptor or on separate biomolecules within an assembly. In the lattermost case, photoreactive crosslinking reagents can reveal interactions among proteins, nucleic acids and membranes in live cells. Illumination (usually at <360 nm) of certain photoreactive groups (i.e., aryl azides) generates reactive intermediates that form bonds with nucleophilic groups.

Benzophenone derivatives, such as the benzoylbenzoic-dUTP described can be repeatedly excited at <360 nm until they generate covalent adducts, without loss of reactivity. Benzophenones generally have higher crosslinking yields than the aryl azide photoreactive reagents (Dorman & Prestwich, 1994, Biochemistry 33: 5661). The succinimidyl ester of 4-benzoylbenzoic acid (Molecular Probes product #B-1577) and benzophenone isothiocyanate (B-1526) have proven useful for synthesizing photoreactive peptides (see, e.g., J. Virol. 38: 840 (1981); J. Protein Chem. 3: 479 (1985); Proc. Natl. Acad. Sci. U.S.A. 83: 483 (1986); and Biochemistry 32: 2741 (1993) and oligonucleotides (see, e.g., Nucleic Acids Res. 26: 1421 (1998); and Bioconjug. Chem. 10: 56 (1999); see also Nucleic Acids Res. 2000: 28(21), 4382-4390 and Mol. Cell. Biol. 11: 5181-5189 (1991), each of which is incorporated herein by reference).

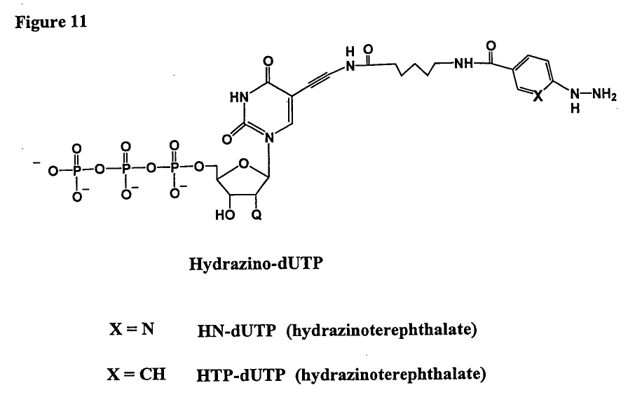

The hydrazino functional group (—NH—NH₂) is another example of a functional group useful according to the invention. Hydrazine derivatives react with ketones and aldehydes to yield stable hydrazones. Hydrazine derivatives also have amine-like reactivity and can be coupled to water-soluble carbodiimide-activated carboxylic acid groups in drugs, peptides and proteins or to carbohydrates following oxidation with sodium periodate. A hydrazino-modified nucleotide is schematically depicted in FIG. 11.

Phosphate Groups

The “Phosphate” moiety can be a mono-, di-, tri- or tetra-phosphate.

Linkers Useful According to the Invention

The linker moiety can be attached to the nucleobase at any position that does not interfere with the ability of the nucleobase to participate in Watson-Crick base pairing. Positions that do not interfere with Watson-Crick base pairing are generally those that do not participate in the internucleobase hydrogen bonding characteristic of Watson-Crick base pairing. For example, linker arm attachment at the N-4 or C-5 position of pyrimidines (or a position spatially equivalent to these positions in a pyrimidine analog) is acceptable. The linker arm can be attached to purines at either N-6, C-8 or C(N)₇. dATP and dCTP are generally modified at the C6 position and the C4 position of the nucleobases, respectively. These sites do participate in hydrogen bonding in the heteroduplex, which makes them less attractive as sites for linker-mediated labeling. When an alternative ring system is chosen (such as pyrazolo[3,4-d]pyrimidine) the linker should be positioned to be structurally equivalent to the acceptable positions on a purine or pyrimidine nucleotide.

The linker can consist of any of a variety of structures and can vary considerably in length. Preferably, the backbone of the linker (the straight chain portion) contains 1 to 50 atoms. Suitable linkers for use in nucleotides according to the invention include those described in U.S. Pat. Nos. 5,047,519 and 5,151,507, and in WO 96/11937, each of which is incorporated herein by reference. Examples include the following:
—CH₂—(CH₂—CH₂)ᵥ—CH₂—NHC(O)-Q-; —CH₂—(CH₂—CH₂)ᵥ—CH₂—C(O)—NH—C(O)-Q-; —S—CH₂C(O)-Q-; —S—CH₂CH₂NH—C(O)-Q-; —O—CH₂C(O)-Q; —O—CH₂CH₂NH—C(O)-Q-; —NH—(CH₂)ᵥ—NH—C(O)-Q-; 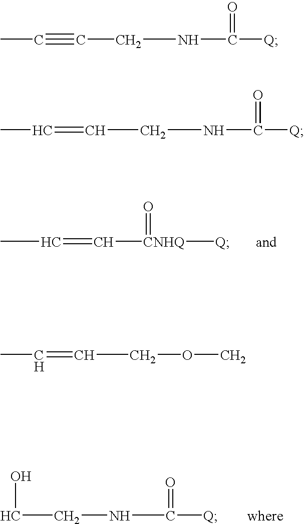
v=0,1,2,3 and
Q=—NH(CH₂)₆NH—, —NH—(CH₂)₂—NH, —(CH₂)₅NH—,
   =—(CH₂)₂—C(O)—NH—(CH₂)₃—O—(CH₂)₂—O—(CH₂)₂—O—(CH₂)₃—NH—,
   =—NH—[(CH₂)₂—O—)_{w}—(CH₂)₂—NH—, w=2,3,4,5,
   =—(CH₂)₂ C(O)—NH—[(CH₂)₂—O]_{w}—NH—, w=2,3,4,5.

A portion of the linker can also contain a carbocyclic (or heterocyclic) structure to effect rigidity. One example is a cyclohexyl component as described in Helvetica. Chim. Acta, 1999, 82: 1311-1323; see also the MCC-modified analogs described herein, which comprise a cyclohexyl group in the linker.

One skilled in the art and having the benefit of this disclosure will appreciate that many alternative linker structures may be utilized in the invention, as long as they are able to join a given functional group to a nucleotide without substantially altering the base-pairing relationships of the nucleotide. In this context, “substantially altering” means that the relative preference of the nucleotide for base pairing with a particular complementary nucleotide or set of nucleotides is changed from the usual preference of that nucleotide or set of nucleotides. If, for example, the addition of a linker on A changes the usual preference of A pairing with T such that A now base pairs with C or does not base pair with any nucleotide, the relative preference for base pairing has changed from the usual preference for that nucleotide, and the base pair relationship is “substantially altered.”

In addition to attachment of the desired moiety to the modified nucleotide, the linker functions as a spacer that positions the attached moiety at a sufficient distance to avoid steric hinderance problems. The effects of linkers attached to deoxyuridine (dU) residues on oligonucleotide hybidization is described in Bull. Chem. Soc. Jpn 1995, 68: 1981-1987. The effects described provide guidance to one skilled in the art regarding the design and placement of linkers onto dU residues such that they continue to permit oligonucleotide hybridization.

Nucleobases Useful According to the Invention

Nucleobases useful according to the invention include a purine, a 7-deazapurine, a pyrimidine, or any nucleobase analog that permits the enzymatic incorporation of the nucleotide analog comprising that nucleobase analog, and is capable of forming Watson-Crick base pairs with a nucleobase on an adjacent antiparallel nucleic acid strand. A measure of whether a nucleobase analog forms a Watson-Crick base pair with a nucleobase on an adjacent polynucleotide strand is whether a nucleotide comprising that nucleobase analog is incorporated into a polynucleotide by a template-dependent nucleic acid polymerase as described herein. In a preferred embodiment, the nucleobase is selected from the group consisting of: adenine, cytosine, guanine, thymine, uracil, hypoxanthine, inosine, 7-deazapurines, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine. Other nucleobases useful according to the invention include, but are not limited to 5-methylcytosine (5-me-C), 5-hydroxymethyl cytosine, xanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine, 3-deazaadenine, and pyrazolo[3,4-d]pyrimidine.

Sugars Useful According to the Invention

Sugar moieties useful according to the invention include any sugar moiety as defined herein that permits the enzymatic incorporation of the nucleotide or nucleotide analog comprising that sugar into a nucleic acid strand. Sugar moieties specifically include, among others, both deoxyribofuranosyl sugars and ribofuranosyl sugars. The sugar moiety is a moiety which occupies a position in the nucleotide analog relative to the other components of the nucleotide analog which is equivalent to the position occupied by the pyrofuranose sugar ring in a traditional ribo- or deoxyribonucleotide. The sugar moiety can be, for example, ribofuranose, 2′-deoxyribofuranosyl, 2′,3′-dideoxyribofuranosyl, phosphonomethoxyethyl, 2-oxyethoxymethyl, 2-hydroxymethoxymethyl, 2-methoxy-3-oxapentanol, 3-pentenyl, oxetan, pyran or oxadiazepine. Additional sugar moieties or non-sugar groups that substitute for the sugar moiety are described, for example, in Bioorg. Med. Chem. Lett. (1997) 7: 3013-3016, Nucl. Acids Res. (1999) 27: 1271-1274, and Nucleosides and Nucleotides (1993) 12: 83-93, each of which is incorporated herein by reference. In one embodiment, the sugar moiety is a cyclic, non-furanose sugar. Examples of cyclic, non-furanose sugars include, but are not limited to oxetan, pyran, or oxadiazepine (see below: P is a mono-, di-, tri- or tetraphosphate; Base is a nucleobase as the term is defined herein):

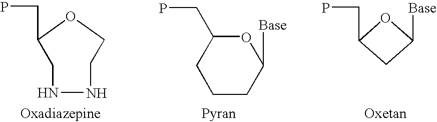

Acyclic sugar moieties useful according to the invention have the general structure shown below: 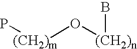
m=an integer from 0-2
n=an integer from 0-3
Non-limiting examples of acyclic sugar moieties useful according to the invention include phosphonomethoxyethyl, 2-oxyethoxymethyl, 2-hydroxymethoxymethyl, and 2-methoxy-3-oxapentanol.
How to Make the Modified Nucleotides of the Invention:

Materials and methods generally useful for the synthesis and purification of the modified nucleotides according to the invention are detailed as follows.

Reagent chemicals and solvents are obtained from Aldrich (Milwaukee, Wis.) unless otherwise noted. Amino-4-UTP was obtained from Sigma (St. Louis, Mo.). Succinimidyl-3-(2-pyridyldithio)propanoate (SPDP) was obtained from Molecular Probes (Eugene, Oreg.). Succinimidyl-4-(N-maleimido-methyl) cyclohexane-1-carboxylate (SMCC) and sulfonated, long chain SPDP (sulfo-LC-SPDP) were obtained from Pierce Chemical Co. (Rockford, Ill.). FLASH™ and Illuminator™ detection kits, DNA polymerases (Taq, T-7, and Pfu), T4 poynucleotide kinase (PNK), terminal transferase (TdT) reaction buffers and naturally-occurring nucleotides were obtained from Stratagene (La Jolla, Calif.). The redundant term, dTTP, is used herein as an abbreviation for the deoxyribonucleotide, thymidine triphosphate.

Nucleotide coupling reactions were monitored by analytical reversed phase HPLC. This system consisted of two Shimadzu LC600 pumps monitored by a SPDM6A photodiode array detector, reversed phase column (5μ, 4.6×250 mm, Rainin) and 100 mM triethylammonium bicarbonate (A) and 60/40 acetonitrile/water (B) as solvents. Following injection of 8 μL aliquots from the reaction mixture, the column was eluted at 1 mL/minute for 10 minutes using a mixture of 97.5/2.5% (A/B). The concentration of solvent B was gradually increased to 65% over a 40 minute time interval.

Nucleotide products were isolated by low-pressure ion exchange chromatography or semi-preparative HPLC. The former method utilized a column loaded with DEAE Sepahrose-Fast Flow ion-exchange resin eluted with a gradient of 0-0.8M triethylammonium bicarbonate pumped by a PI-peristaltic pump, collecting 3.5 mL fractions using a Pharmacia FRC 100 fraction collector (all components were obtained from Pharmacia, Uppsala Sweden). The semi-preparative HPLC system contained two Shimadzu 10AS pumps, SPD 10A detector and reversed phase column (7μ, 10×250 mm, S5ODS2, PhaseSep). Multiple injections of the reaction mixture were made using a Shimadzu SIL10A autosampler. Eluate was monitored at 293 nm (Quick Link nucleotides) or 480 nm (fluorescent nucleotides) at 3 mL/minute with the same solvents described for analytical HPLC. Gradient profile: 0-10 minutes, 2.5% B, increased to 35% B at 38 minutes and 100% B at 39-50 minutes. Appropriate fractions were pooled, evaporated to dryness in vacuo, co-evaporated several times with ethanol, resuspended in buffers as described and stored at −20° C.

Synthesis of Modified Nucleotide Analogs

A generally applicable approach to the generation of functionalized nucleotide analogs according to the invention is to react amino-modified nucleotides (e.g., Amino-11-dUTP or -dCTP or 7-deazadATP/dGTP as described by Hobbs, U.S. Pat. No. 5,047,519, incorporated herein by reference) with the appropriate succinimidyl esters of the functional groups to yield the modified nucleotides. That is, the amino modification on the nucleotide is reacted with a succinimidyl group on a molecule that carries the functional group one wishes to append to the nucleotide.

Examples of the synthetic approaches and conditions for modified nucleotide analogs according to the invention are provided below. In the following description, the nucleotide receiving the modifying group is dUTP or UTP, however, it should be understood that any nucleotide meeting the Phosphate-Sugar-Nucleobase-Linker-F formula can be modified by one of skill in the art to contain the functional groups useful according to the invention.

a) SAc-dUTP

To a stirring solution of amino-11-dUTP (10 mg, 8.8 μmole) in 3 mL of 100 mM sodium borate (pH 9) was added dropwise a solution of N-succinimidly-S-acetylthio-acetate (22 mg, 8.8 μmole) in 500 μL of dimethylformamide and the reaction mixture was stirred for 2 hours at room temperature. Analytical HPLC analysis revealed the formation of a new product (elution time 23 minutes) which was isolated by semi-preparative HPLC as described above.

b) SAc-UTP

Amino-4-UTP (10 mg, 8.8 μmole) in 3 mL of 100 mM sodium borate (pH 9) was combined with 20 mg N-succinimidly-S-acetylthio-acetate (22 mg, 8.8 μmole) in 500 μL of dimethylformamide and the reaction mixture was stirred for 2.5 hours at room temperature. Analysis of a reaction aliquot using analytical HPLC showed the formation of a new product which exhibited a retention time of 33.2 minutes. This product was isolated by semi-preparative HPLC as described above and stored in 100 mM Tris, pH 7.4.

c) Di-S-methyl-triazine-dUTP

Synthetic steps for generating a di-S-methyl-triazine-modified nucleotide (di-S-methyl-triazine-dUTP) are shown schematically in FIG. 9.

d) Benzoylbenzoic-dUTP

An example of the synthetic conditions for generating a nucleotide bearing a benzoylbenzoic functional group is as follows. Amino-11-dUTP is reacted with succinimidyl benzoylbenzoic acid (B1577, Molecular Probes) in 100 mM sodium borate, pH 9. The reaction is monitored by analytical HPLC and the product, Benzophenone-12-dUTP is purified by preparative, reversed-phase HPLC.

e) Hydrazino-dUTP

An example of the synthetic conditions for generating a nucleotide bearing a hydrazino functional group is as follows. Amino-11-dUTP is reacted with succinimidyl 6-hydrazinonicotinate or succinimidyl 6-hydrazinoterephthalate (TriLink Biotechnologies) in 100 mM sodium borate, pH 9. The reaction is monitored by analytical HPLC and the product, HN-12-dUTP (hydrazinonicotinate) is purified by preparative, reversed-phase HPLC.

How to Use Modified Nucleotides According to the Invention:

Modified nucleotides according to the invention and nucleic acids comprising them are useful both for the attachment of detectable moieties or affinity reagents and for the attachment of the nucleotides or nucleic acids to surfaces or supports. For example, modified nucleic acids can be reacted with fluorescent dyes, enzymes, antibodies, epitopes or members of a specific binding pair containing complementary reactive groups. Alternatively, it can be useful to covalently attach a functionalized DNA or RNA sequence to a transcription factor that recognizes that sequence, either simply to label it or to facilitate studies of the protein:nucleic acid interaction. Attachment to a solid support can include covalent attachment of a nucleic acid to, e.g., plates, tubes, beads or column matrices. A non-exhaustive list of examples of commercially available products useful for reaction with maleimide-, PDP- or SAc-modified nucleic acids are provided in Table 1. Once the functionalized nucleotide is incorporated into the nucleic acid, the functionalized nucleic acid can be reacted with detectable moieties, polypeptides or solid supports that bear the complementary reactive group for the functional group.

Labeling Nucleic Acids:

A) Incorporation of Modified Nucleotides.

Modified nucleotides according to the invention are enzymatically incorporated into nucleic acid probes in the same manner as standard nucleotides. Thus, they can be incorporated by nucleic acid polymerases and by enzymes such as terminal deoxynucleotidyl transferase (TdT). Non-limiting examples of useful polymerases include DNA polymerases, such as the Klenow fragment of E. coli DNA polymerase, Taq polymerase or other thermostable DNA polymerases, RNA polymerases, such as T7 or T3 polymerase, and reverse-transcriptases, such as AMV and MMLV reverse transcriptases.

Conditions for enzymatic labeling reactions are well known to those skilled in the art and will vary with the enzyme and with the template (e.g., RNA vs. DNA, single-stranded vs. double stranded). Enzymatic labeling reactions include nucleic acid template, appropriate buffer, enzyme, and functionalized nucleotide. Depending upon the type of reaction (e.g., end labeling versus body labeling), it can be necessary to include non-functionalized nucleotides, and it will often be desirable to include both a standard nucleotide and the modified form of that nucleotide (e.g., dA and functionalized dA) in the same reaction. The presence of the modification will preferably not affect the efficiency of enzyme recognition or incorporation, but enzymes will frequently exhibit at least some bias for or against the functionalized nucleotides. One skilled in the art can adjust the overall concentration of the functionalized nucleotide or nucleotides, as well as the ratio of functionalized to non-functionalized nucletides, in order to achieve optimal labeling results. Examples 1 and 4, below, describe various ways to incorporate a functionalized nucleotide according to the invention.

Incorporation of a functionalized nucleotide according to the invention results in a functionalized nucleic acid molecule. Standard means known in the art, such as size-exclusion chromatography, gel purification and/or precipitation can be used to purify the labeled nucleic acid away from unincorporated nucleotides.

B) Reaction of Functionalized Nucleic Acids with Targets.

Following the removal of unincorporated nucleotides, the functionalized nucleic acids are reacted with targets containing reactive groups complementary to the functional group or groups on the nulceic acid. Thus, fluorescent or chromogenic dyes, polypeptides, enzymes, antibodies, epitopes or members of a specific binding pair, each comprising a complementary reactive group, are contacted with the functionalized nucleic acid under conditions appropriate for the given functional group/reactive group reaction. Such conditions are known to those of skill in the art. Examples of conditions for exemplary specific functional groups are described herein above. Similar approaches are taken for the attachment of nucleic acids to surfaces or supports bearing complementary reactive groups. In each instance, depending upon the exact nature of the linkage reaction, there may be a requirement for treatment of the functionalized nucleic acid or the support in order to expose or deprotect a functional group. These pre-treatments are well within the grasp of one skilled in the art. Examples 2, 3 and 4, below, detail the labeling of a functionalized nucleic acid according to the invention.

It can be useful in some instances to label nucleotides directly, by reaction of the functionalized nucleotide with a target, for example a dye. In such instances, the reactions can be conducted in the same manner, although, depending upon the target, purification may need to be altered. For example, the attachment of a fluorecent dye to a nucleotide may require purification by HPLC to remove labeled from unlabeled nucleotide.

While functionalized nucleotides can be labeled before incorporation, it will generally be preferable to incorporate the functionalized nucleotides and then react with target. This approach avoids possible interference of the label with the efficiency of the incorporating enzyme.

How to Use Nucleic Acids Labeled According to the Invention

Nucleic acids labeled as described herein can be used in essentially any process or assay calling for labeled nucleic acids. A primary use is for hybridization analyses, including, for example, Northern, Southern and dot-blot analyses, as well as in situ hybridization analyses. In general, standard hybridization conditions will apply, because the addition of label on a linker attached to the nucleobase will not dramatically alter the hybridization kinetics or stability of the hybridized complex. This is especially true where care has been exercised to place the label on the nucleobase at a site that does not interfere with the hydrogen bonding necessary for Watson-Crick base pairing. In this sense, there may be some advantage to using longer, instead of shorter linker molecules, because the label will be separated from the backbone of the nucleic acid, reducing chances for interference with hybridization.

Examples 3 and 5, below, detail the use of a non-isotopically labeled probe according to the invention in Southern and dot blot hybridization assays to detect human IL-2 and actin DNA and mRNA. In situ hybridization is described in Example 6, below.

Functionalized nucleotides can be used for labeling nucleic acids during PCR amplification. The concentration and ratios of functionalized nucleotides can be optimized by one skilled in the art. Deoxynucleotides containing very long linker arms have been reported to be good substrates for Taq and Vent™ DNA polymerases. (Zhu, et al., 1994, Livak, Hobbs and Zagursky,1992). Example 4, below, details PCR labeling using functionalized nucleotides.

Other uses for labeled nucleotides made according to the invention include, for example, end-labeling of oligonucleotides for sequencing analysis. While the conditions for the sequencing reactions may require some adjustment, e.g., with respect to the concentration of labeled primer, these adjustments can be made empirically with a minimum of experimentation, and would be well within the grasp of the skilled artisan.

Additional uses and targets of attachment of functionalized nucleic acids are listed in Table 1.

### EXAMPLES

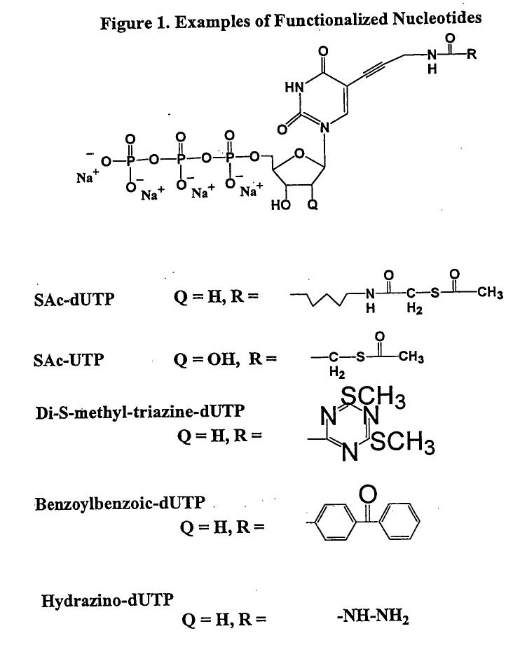

Each of the described nucleotides is analyzed by physico-chemical methods to determine the structure of the isolated products. Analysis of the nucleotides by a combination of UV, HPLC and mass spectrometry is used to confirm that the structure of each nucleotide is as depicted, for example, in FIG. 1. The polymerase-catalyzed primer extension assay is used as an additional confirmation of structure and to determine if the nucleotides would function as substrates for DNA polymerases.

The functionalized nucleotide analogs were tested for their incorporation into short DNA fragments using terminal deoxynucleotide transferase and using Klenow, exonuclease free Klenow, T7 and Taq DNA polymerases (see Appendix 1).

### Example 1

A. Tailing with Terminal Deoxynucleotide Transferase (TdT)—“Procedure 1”:

A 12-mer oligonucleotide (5′-CCTGGTCGTCGG-3′; SEQ ID NO: 1) was 5′ labeled with ³²P ATP and T4 Polynucleotide Kinase according to established methods (Roychoudhury and Wu, 1980; Sambrook, Fritch, and Maniatis, 1989). Aliquots of the mixture containing 10 ng of kinased oligo were combined with 100 pmol of fluorescein 12-dUTP and 5 units of terminal deoxynucleotide transferase (TdT) in 100 mM potassium cacodylate, 2 mM CoCl₂, pH 7.2 (final volume of 10 μL). The reaction mixtures were incubated at 37° C. for 10 minutes. The reactions were quenched by the addition of loading dye and 4 μL aliquots were loaded onto a sequencing gel (14″×17″, poured with 20% acrylamide/7M urea/1×TBE), electrophoresed for 3 hours at 55 watts/2250 mV and exposed to X-ray film. A measurement of the degree of polymerase-catalyzed primer extension obtained in the presence of other modified nucleotides can be determined by substituting each modified nucleotide (1 μL of 100 μM) for fluorescein 12-dUTP in this procedure (see, e.g., Appendix 1).

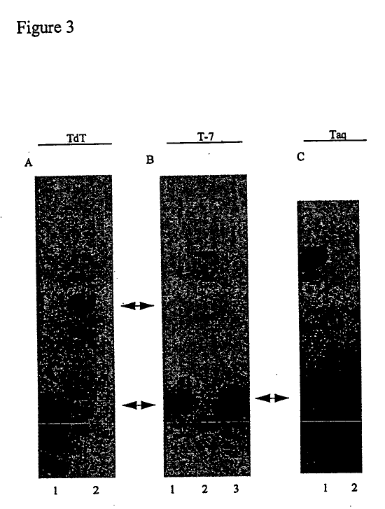

PDP-dUTP proved to be a substrate for TdT under the normal conditions for tailing of oligonucleotides (procedure1). Multiple additions of the modified nucleotide to the 3′ terminus of the starting oligonucleotide were observed on a 20% denatured PAGE gel (see FIG. 3A). Bands were observed which corresponded to the addition of up to 10 contiguous PDP-dUTP bases. In a separate assay, up to 5 base additions were observed in TdT-catalyzed oligonucleotide end labeling in the presence of MCC-dUTP. The amount of end labeling appeared to be proportional to the amount of enzyme in the reaction and less dependent on the concentration of MCC-dUTP (results not shown). Similar methods are applicable for nucleotides bearing any of the functional groups according to the invention.

B. Polymerase Extension Assay—“Procedure 2”:

Aliquots containing 10 ng of the kinased 12-mer described in procedure 1 were combined with 40 ng of an 18-mer oligonucleotide (5′-ATAATACCGACGACCAGG-3′; SEQ ID NO: 2) in 8 μL of T-7 buffer (20 mM MgCl₂, 50 mM NaCl, 40 mM Tris, pH 7.5) or Klenow buffer (5 mM MgCl₂, 4 mM DTT, 35 mM Tris, pH 7.5). To each reaction was added 1 μL of a solution containing 100 μM each of dATP and fluorescein 12-dUTP. The reaction contents were heated to 95° C. for 2 minutes and allowed to cool to room temperature. Five units (1 μL) of the appropriate polymerase were added and the reaction mixtures were incubated at 37° C. for 10 minutes. Control reactions were run which contained no polymerase. Polymerase activity was terminated by addition of 3 μL of 1M EDTA and the extension products were separated by electrophoresis using a 20% acrylamide gel and the results visualized by autoradiography. A measurement of the degree of polymerase-catalyzed primer extension obtained in the presence of modified nucleotide analogs according to the invention can be determined by substituting the modified nucleotide (1 μL of 100 μM) for fluorescein 12-dUTP in procedure 2.

Functionalized nucleotide MCC-dUTP was accepted as a substrate for Klenow and exonuclease free Klenow polymerases when tested in a primer extension assay (procedure 2, results not shown). Similarly, nucleotide PDP-dUTP was incorporated into DNA by T7 and Taq DNA polymerases during primer extension reactions (procedure 2, see FIGS. 3B and 3C). The sequence of the template used in this assay was configured so that dTTP/dUTP analogs would be incorporated into (a maximum of) four sites. The size of the major reaction products made in the presence of Taq polymerase and PDP-dUTP indicated that the modified nucleotide was incorporated at several sites. Analysis of the reaction products by denaturing polyacrylamide electrophoresis showed a band which was common to both reactions, i.e., T7 and Taq. In the T7 reaction, bands corresponding to smaller sized fragments were also observed which indicated that the modified nucleotide exhibited reduced substrate efficiency with T7 polymerase compared to Taq polymerase. Nucleotide MCC-dUTP was similarly tested with Taq and T7 polymerases and showed essentially the same degree of incorporation as PDP-dUTP. Similar methods are applicable for nucleotides bearing any of the functional groups according to the invention.

C. Polymerase Extension Assay—“Procedure 3”:

For dUTP analogs, aliquots containing 10 ng of kinased 17-mer oligonucleotide (5′-CCTGGTCGT-CGGCGTAC-3′; SEQ ID NO: 3) were combined with solutions containing 100 ng of 63-mer (5′-GCTTACCAGTCATCGGGTCCAAGTGTATAGACGCATGAGAGTGTA-GGTACGCCGACGACCAGG-3′; SEQ ID NO: 4), 10 μM (each) dGTP, dATP, dCTP and modified dUTP, in either T7 buffer (20 mM MgCl₂, 50 mM NaCl, 40 mM Tris, pH 7.5), Klenow buffer (5 mM MgCl₂, 4 mM DTT, 35 mM Tris, pH 7.5) or Taq buffer (1.5 mM MgCl₂, 50 mM KCl, 0.001% gelatin, 10 mM Tris, pH 8.5). Five units of the appropriate polymerase were added to bring the final reaction volume to 10 μL. The reaction mixtures were incubated at 37° C. (Klenow and T7 reactions) or 72° C. (Taq reactions) for 10 minutes. Control reactions were performed using H₂₀ or 10 μM dTTP in place of the modified nucleotide. Polymerase activity was terminated by addition of 3 μL of 1M EDTA and the extension products were separated by electrophoresis using a 20% acrylamide gel and the results visualized by autoradiography.

When modified dCTP analogs were tested, the nucleotide mixes included 10 μM (each) dGTP, dATP, dTTP and modified dCTP. Positive control reactions were performed using 10 μM dCTP in place of the modified nucleotide. Negative control reactions were performed using H₂O in place of the modified nucleotide. When modified dATP analogs were tested, the nucleotide mixes included 10 μM (each) dGTP, dCTP, dTTP and modified dATP. Control reactions were performed using H₂₀ or 10 μM dATP in place of the modified nucleotide.

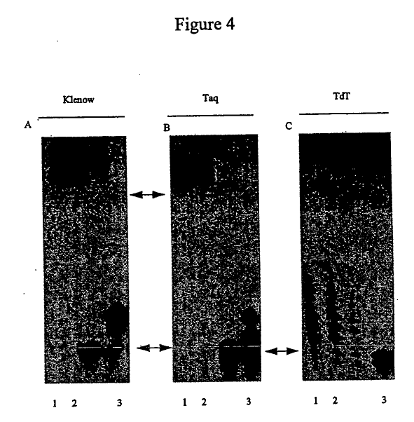

Results of primer extension reactions using SAc-dUTP according to procedure 3 showed that the nucleotide was efficiently incorporated by Klenow and Taq DNA polymerases (see FIGS. 4A, B). Two different lots of SAc-dUTP were tested (fractions 1 and 2). In each case, one major product was formed. The band corresponding to the major product migrated slightly slower than a 63-mer marker (indicated by the top arrow). Altered gel migration rates would be expected for DNA containing modified nucleotides. Polyacrylamide gel analysis of nucleic acid products made by enzymatic incorporation of modified nucleotides compared to those made with unmodified nucleotides demonstrated that DNA products containing modified nucleotides migrate slower than products of equivalent length containing only unmodified nucleotides. In this light, the results obtained using SAc-dUTP infer that full-length extension products were made with incorporation of the modified nucleotide at twelve sites (63-mer template, procedure 3). Analog SAc-dUTP was also accepted as a substrate for TdT in the 3′-end labeling assay (Procedure 1) as shown in FIG. 4C. Similar methods are applicable for nucleotides bearing any of the functional groups according to the invention.

### Example 2

DNA Probe Generation/Detection Using the Functionalized Nucleotide, MCC-dUTP—“Procedure 4”:

Oligonucleotides were end labeled using the functionalized nucleotide MCC-dUTP, and subsequently conjugated with modified alkaline phosphatase to form an alkaline phosphatase-tailed oligonucleotide which was used as a hybridization probe. The thiol-protected nucleotide analogs, PDP-dUTP and SAc-UTP, were also used to generate tailed oligonucleotides.

Fifteen micrograms of a 40-mer oligonucleotide homologous to a region of the human Interleukin 2 gene were combined with solutions containing 2.7 mM MCC-dUTP (1 and 2 μL) and 15 units of TdT in 100 mM potassium cacodylate, 2 mM CoCl₂ (pH 7.2). The contents were incubated for 15 minutes at 37° C. The reaction mixtures were individually loaded onto Nuc Trap™ purification columns and eluted with 100 μL of 100 mM NaCL-10 mM Tris HCl(pH 7)-1 mM EDTA. The first 120 μL was collected and stored at −20° C.

In a separate reaction vessel, 3 mg of alkaline phosphatase was added to 175 μL of 100 mM sodium phosphate, 1 mM 2-mercaptoethanol (pH 8.0) and combined with 25 μL of 2-iminothiolane (Traut's reagent, 200 mM in the same buffer). The reaction mixture was vortexed briefly, left at room temperature for 30 minutes and afterwards loaded onto a Pharmacia PD-10 drip column equilibrated in 100 mM sodium phosphate (pH 7.3). The column eluate was collected in 500 μL fractions. Appropriate fractions (6-8) were pooled and stored at 4° C. until further use.

Aliquots (corresponding to 300 μg) from the thiol-modified alkaline phosphatase mixture were combined with each of the MCC-dUTP end labeled oligo mixtures described above and allowed to incubate at room temperature for two hours. The mixtures were concentrated to a volume of 50 μL using a Centricon™ 305 spin column (4500 rpm at 4° C.), combined with 7 μL of sterile glycerol and loaded onto a 6% acrylamide gel which was electrophoresed at 300V and 4° C. in 1 mM DTT-1×TBE until the bromophenyl blue dye ran 2/3 of the length of the gel. Gel fractionated products were visualized by UV shadowing techniques which revealed two bands that migrated approximately 2 cm from the loading well. The slower migrating band migrated equivalent to a sample of unconjugated alkaline phosphatase. The faster migrating band, assumed to be oligo-enzyme conjugate, was excised from the gel and combined with 2 mL of 10 mM Tris (pH 7.5), 5 mM MgCl₂, 0.1 mM ZnCl₂, 0.002% sodium azide, 50% glycerol. After 12 hours, aliquots (25 μL) of the dialyzed gel slice solutions were used as hybridization probes.

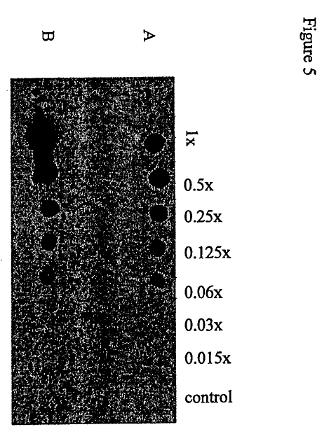

Serial dilutions of heat denatured target DNA (PCR amplification of the third exon of the human Interleukin 2 gene) were spotted onto Illuminator™ nylon membrane, UV crosslinked and prehybridized in Quick-HYB™ at 68° C. for 30 minutes. To the hybridization solution was added 25 μL of the oligo-MCC-alkaline phosphatase probe and the mixtures incubated at 48° C. for one hour. The membrane was washed twice at 50° C. in 2×SSPE-1% SDS for 10 minutes each and then treated with a solution of 0.1M diethanolamine-1 mM MgCl₂-0.02% sodium azide for 5 minutes at room temperature. The membrane was treated with a solution of 0.1M diethanolamine-1 mM MgCl₂-0.02% sodium azide-56 μg/mL 4-methoxy-4-(3-phosphinicophenyl)-spiro[1,2-]dioxetane-3,2′adamantane (PPD) for 5 minutes at room temperature and covered with plastic wrap and exposed to X-ray film for 40 minutes (see FIG. 5).

FIG. 5 shows serial dilutions of oligonucleotide-alkaline phoshatase probes that were made by coupling thiol-modified alkaline phosphatase to oligonucleotides that had been end labeled with MCC-dUTP. The resultant alkaline phosphatase-tailed oligonucleotide probes were used to sucessfully detect the second exon of the human IL2 gene in a dot blot format. The level of sensitivity was not determined in this experiment. These results verified earlier results indicating that nucleotide MCC-dUTP could be sucessfully incorporated at the 3′ termini of oligonucleotides and also showed that the end labeled oligos could be successfully coupled to thiol-modified alkaline phosphatase. The analog, SAc-UTP, was also used to generate alkaline phosphatase-labeled riboprobes which were suitable for the detection of membrane-immobilized target DNA (results not shown). Nucleic acids labeled using other functional groups described herein can be used in a similar manner.

### Example 3

DNA Probe Generation/Detection Using the Functionalized Nucleotide, PDP-dUTP—“Procedure 5”:

The invention provides nucleotides including 5-pyridyl-dithiolpropyl (PDP)-modified nucleotides and 5-S-acetyl thioethyl (SAc)-modified nucleotides. These nucleotide analogs contain protected thiol groups which are useful for attachment to a variety of materials containing maleimides and haloacetyl functionalities. Enzymatic labeling of nucleic acid with these analogs produced nucleic acids modified with reactive thiol groups which were subsequently coupled to modified alkaline phosphatase. Additional materials that could be coupled to DNA derivatized with PDP or SAc include maleimide derivatized polystyrene tubes (Covalink™+SMCC, Nunc, Naperville, Ill.), acrylamide beads (bromoacetyl BioGel, BiORad; Trisacryl GF2000, IBF Corp; Fahy, et al., 1993), tresyl and epoxy resins (Toyopearl®, TosoHaas), and others as listed in Table 1.

Oligos targeting the third exon of the IL2 gene and, separately, oligos homologous to a mouse actin gene were 3′-end labeled using PDP-dUTP and terminal transferase analogous to the end labeling reaction using MCC-dUTP, described in procedure 4. The reactions were treated with 20 μL of 1M DTT (to unmask the thiol groups on the nucleotides), vortexed briefly and the mixture loaded onto a 1.8 mL Bio-Gel P-60 column equilibrated in 10T.1E and eluted with 300 μL of the same buffer. An additional 400 μL of 10T.1 E was added and the resulting eluent collected, lyophilized to dryness and resuspended in 200 μL of 0.1 M sodium phosphate (pH 6.0). In a separate reaction, a solution containing 3 mg of alkaline phosphatase in 1 mL of 100 mM sodium borate (pH 8.5) was treated with 5 mg of SMCC and the mixture was gently mixed for one hour at room temperature. The maleimide-modified alkaline phosphatase was then purified using a PD-10 drip column equilibrated in 100 mM sodium phosphate (pH 6.5) following the method described for thiol-modified alkaline phosphatase in procedure 7. Aliquots containing ≈300 ng of maleimide-modified alkaline phosphatase in 300 μL of 100 mM sodium phosphate (pH 6.5) were combined with the deprotected PDP-dUTP end labeled oligos, vortexed briefly and allowed to stand for two hours at room temperature. The oligo-PDP-alkaline phosphatase probes were concentrated and gel isolated as described in procedure 4.

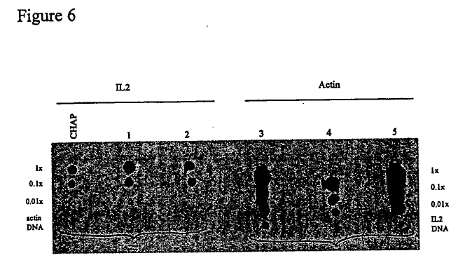

Serial dilutions of heat denatured target DNA (IL2 gene and pActin A) were immobilized onto Illuminator™ nylon membrane as described above and prehybridized in 5 mL of Quick-HYB™ at 50° C. for 20 minutes. Ten microliters of each dialyzed probe solution were added and the mixtures were incubated at 50° C. for one hour. The membrane was washed and treated according to the detection protocol described in procedure 4. Results are shown in FIG. 6.

FIG. 6 shows that the functionalized nucleotide PDP-dUTP was successfully used according to procedure 5 to end label oligonucleotides that were subsequently coupled with maleimide-modified alkaline phosphatase. The oligo-alkaline phosphatase conjugates were used as hybridization probes for the detection of the IL2 gene (FIG. 10, columns 0-2, rows 1-3) and actin genes (columns 3-5, rows 1-3) on a dot blot format. Under the low stringency washing conditions described, nonspecific signal was observed with the actin probe (columns 3-5, row 4). These results demonstrate, however, that suitable nonradioactive probes were made from PDP-dUTP-tailed oligonucleotides after coupling with maleimide-modified alkaline phosphatase. Alkaline phosphatase or other enzymes linked to nucleic acid probes via other functional groups described herein can be used in a similar manner.

### Example 4

PCR Labeling of DNA Using Functionalized Nucleotides—“Procedure 6.”

PDP-dUTP is included in PCR amplification reactions, followed by the reaction of the functionalized DNA with fluorescein-5-maleimide.

Into separate PCR amplification vessels are added 5 uL of Taq reaction buffer, 20 ng of Bluescript KS+, 125 ng (each) of reverse and −20 primers, or 125 ng (each) of M13 −20 primer and 066 primer (5′-GCTAATCATGGTCATAGCTGTT-3′; SEQ ID NO: 5), 7.5 uL of a 1 mM dGTP-dATP-dCTP solution and 7.5 uL aliquots containing either 500 μM dTTP-500 μM PDP-dUTP or 250 μM dTTP-750 μM PDP-dUTP. Control reactions contain 1 mM dTTP in place of the dTTP/PDP-dUTP mixtures. Reaction vessels are placed in a thermal cycler and treated according to the following cycling parameters: initial denaturation at 94° C. for 45 seconds followed by 28 cycles of 94° C. for 45 seconds, annealing at 50° C. for 1 minute and extension at 72° C. for 1 minute 15 seconds.

The PCR reaction contents containing 220 bp and 550 bp amplicons are treated with DTT to deprotect and reveal the thiol groups, then purified using Nuc trap™ columns using water in place of TBS according to the protocol provided. The collected samples are lyophilized to dryness and resuspended in 100 mM sodium phosphate (pH 6.0). Aliquots of deprotected, functionalized DNA are combined with fluorescein-5-maleimide dye (Molecular Probes, Eugene, Oreg.) in 100 mM sodium phosphate (pH 6.5) and allowed to stand for two hours at room temperature to generate fluorescein-labeled DNA. Nucleotides bearing other functional groups can be labeled and used in an analogous manner.

The fluorescein-labeled PCR products are then purified by loading the reaction mixture onto a column containing 2 mL of Bio Gel P60, eluting with a low salt buffer (15 mM NaCl and 15 mM Tris buffer, pH 7.5), and collecting the effluent in 125 μL fractions.

### Example 5

Use of Oligonucleotide Probes Containing PDP-dUTP for the Detection of Target Sequences on a Southern Blot—“Procedure 7”:

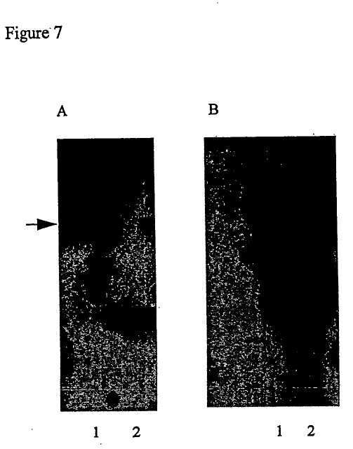

Oligos targeting the third exon of the IL2 gene and, separately, oligos homologous to the mouse actin gene were 3′-end labeled using PDP-dUTP and crosslinked to alkaline phosphatase as described in procedure 5. Aliquots of Eco-R1 digested human genomic DNA were loaded onto an agarose gel, separated by electrophoresis, transferred to a nylon membrane and immobilized by UV crosslinking. The membranes were prehybridized in 2 mL of Quick Hyb™ hybridization solution for 15 minutes at 68° C., combined with 10 μL of the PDP-labeled oligo-alkaline phosphatase probes described above and hybridized at 60° C. for 30 minutes. The membranes were then washed and treated according to the detection protocol described in procedure 4. Results of multiple copy (actin) and single copy (IL2) detection on a Southern blot are shown in FIG. 7.

Analog PDP-dUTP was also used to generate probes by nick translation and random priming methods (results not shown). Oligonucleotide probes end labeled using PDP-dUTP were also used to detect multiple-copy (actin) and single-copy (IL2) genes on a Southern blot. In this experiment, the probe allowed detection of actin genes in 1 μg of EcoR1-digested human genomic DNA (FIG. 7B). The banding pattern in lanes 1 and 2 correspond to the pattern observed in hybridizations with biotinylated oligonucleotide probes (using Flash™ detection). When used for detection of the IL2 target, the probe gave a strong signal (at the site indicated by the arrow) in the lane containing 5 μg of EcoR1-digested human genomic DNA and a weak (but detectable) signal from 1 μg of digested human genomic DNA (FIG. 7A). Oligonucleotides labeled using other functional groups can be labeled and used in an analogous manner.

### Example 6

In Situ Hybridization: Chromosome Painting Using Probes According to the Invention—“Procedure 8”:

The fluorescently labeled probes made according to the invention can be used to generate probes useful for in situ chromosome painting. Probe DNA is generated by PCR using mouse chromosome template DNA (RB 1.3), degenerate oligonucleotide primers and PDP-dUTP essentially as described in Example 4. Following PCR, functionalized DNA is reacted with fluorescein-5-maleimide (Molecular Probes, Eugene, Oreg.) as described in Example 4. The fluorescein-labeled probe is added to a metaphase chromosome spread containing denatured chromosomal DNA and mouse Cot I DNA, hybridized at 37° C. for 24 hours, washed, stained with DAPI/propidium iodide, and viewed under a Nikon fluorescent microscope. Targeted polyploid chromosomes are identified by a bright, evenly-distributed, pink-red signal in proximity to non-target chromosomes that emit a red signal of lesser intensity. Probes labeled using other functional groups described herein can be used in an analogous manner.

### Example 7

Generation and Use of RNA Probes—“Procedure 9”:

Fluorescent riboprobes are made using T3 RNA polymerase and PDP-UTP. Following reaction with maleimide-functionalized fluorescein, these probes are useful for chemiluminescent detection of target RNA and DNA on Northern and Southern blots.

For detection on Southern blots, fluorescein-labeled riboprobes are generated using T3 RNA polymerase, pBluescript® II KS+phagemid template and PDP-UTP following a modification of the procedure described in Stratagene's RNA Transcription kit. A solution containing 1 mM UTP and 1 mM PDP-UTP is substituted for the ³²P-UTP solution and the protocol for the transcription reaction is followed as described. Following transcription and isolation of the transcripts, the functionalized RNA is reacted with maleimide-functionalized fluorescein as described in Example 2.

Aliquots of human genomic DNA are combined with serial dilutions of pBluescript® II KS+ and loaded onto an agarose gel, separated by electrophoresis, transferred to a nylon membrane and immobilized using a UV crosslinker (e.g., the Stratalinker™, Stratagene). The membranes are prehybridized in 2 mL of hybridization solution (e.g., Quick Hyb™, Stratagene) for 15 minutes at 68° C., combined with the fluorescein-labeled riboprobe solution and hybridized at 68° C. for one hour. The membranes ware then washed twice for five minutes at room temperature with 50 mM Tris pH 7.5-150 mM NaCl-0.01% Dowfax 3B2. The blots are treated for 30 minutes with a blocking solution containing 50 mM Tris (pH 7.5)-150 mM NaCl-0.2% Dowfax 3B2, conjugated to an alkaline phosphatase-fluorescein antibody conjugate, washed and incubated with substrate as described in Stratagene's Illuminator™ Nonradioactive Detection protocol. Probe signal on the membranes is detected, for example, using a still video imaging system.

For detection on Northern blots, fluorescein-labeled riboprobes are generated using pBluescript® containing the human alpha 1-antitrypsin insert as template DNA following the procedure described above. Aliquots of total and messenger mouse RNA which contained the human alpha1-antitrypsin transgenic message are loaded onto an agarose gel, separated by electrophoresis, transferred to a nylon membrane and immobilized by UV crosslinking. The membranes are then treated as described above for the Southern blot and the chemiluminescent signal detected using a still video imaging system.

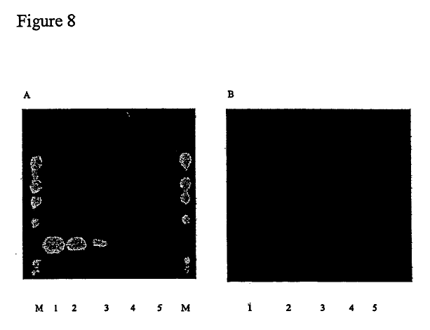

Fluorescent-labeled riboprobes generated using fluorescein-12-UTP have been used to detect 0.5 pg of target pBluescript® DNA on a Southern blot with the Eagle Eye II still video imaging system (see FIG. 8a). In a Northern blot format, riboprobes targeting a portion of human alpha 1-antitrypsin gene were able to detect transcripts in 10 μg of total mouse RNA and 2 μg of transgenic mouse messenger RNA (see FIG. 8b). Similar sensitivities are expected for fluorescent riboprobes made according to the invention. Fluorescent riboprobes prepared using other functional groups according to the invention can be used in an analogous manner.

### Example 8

Reaction of Thioacetyl-Functionalized Nucleic Acid with a Thiol-Reactive Label.

To a S—Ac labeled DNA sample (3 μg) in 50 mM sodium phosphate, 1 mM EDTA, pH 7.5 mL is added 100 μL of deacetylation solution (0.5M hydroxylamine.HCL, 25 mM EDTA, 50 mM sodium phosphate, final pH 7.5). The mixture is vortexed briefly and allowed to stand at room temperature for two hours. Following spin dialysis through a Centricon spin column, the sample is eluted with 50 mM sodium phosphate, pH 7.5 and combined with thiol-reactive label under the conditions described herein for MCC-dUTP. Methods for the use of the S—Ac functional group to couple proteins are known in the art and described, for example, in the following references, which are incorporated herein by reference: Duncan et al., 1983, Anal. Biochem. 132, 68-73; Fuji, et al., 1985, Chem. Pharm. Bull. 33, 362-367; and Ghosh, et al., 1990, Bioconjugate Chem. 1, 71-77.

### Other Embodiments

The foregoing examples demonstrate experiments performed and contemplated by the present inventors in making and carrying out the invention. It is believed that these examples include a disclosure of techniques which serve to both apprise the art of the practice of the invention and to demonstrate its usefulness. It will be appreciated by those of skill in the art that the techniques and embodiments disclosed herein are preferred embodiments only that in general numerous equivalent methods and techniques may be employed to achieve the same result.

All of the literature and patent references identified herein, are hereby expressly incorporated herein by reference to the extent that they describe, set forth, provide a basis for or enable compositions and/or methods which may be important to the practice of one or more embodiments of the present invention.

### REFERENCES

Bernatowicz, M. S., and Matsueda, G. R. Anal. Biochem. 1986 155, 95-102.
Brinkley, M. A., Bioconjugate Chem. 1992, 3, 2-13.
Dirks, R. W., Van Gijlswijk, R. P. M., Vooijs, M. A., Smit, A. B., Bogerd, J., Van Minnen, J., Raap, A. K. and Van der Ploeg, M., Exp. Cell Res., 1991, 194, 310-315.
Duncan, J. S., Weston, P. D., Wrigglesworth, R., Anal. Biochem. 1983, 132, 68-73.
Fahy, E., Davis, G. R., DiMichele, L. J., and Ghosh, S. S., Nuc. Acids Res. 1993, 21, 1819-1826.
Fujita, K. and Silver, J., Biotechniques, 1993, 14, 608-617.
Hobbs, F. W., J. Org. Chem., 1989, 54, 3420-3422.
Igloi, G. L. and Schiefermayr, E., Biotechniques, 1993, 15, 486-495.
Jablonski, E., Moomaw, E. W., Tullis, R. H., and Ruth, J. L., Nucl. Acids Res., 1986, 14, 6115-6128.
Kossel, H and Roychoudhury, R., Eur. J. Biochem., 1971, 22, 271-276.
Kumar, A., Tscen, P., Roullet, F. and Cihen, J., Anal. Biochem.,1988, 169, 376-382.
Livak, K. J., Hobbs, F. W., and Zagursky, R. J., Nucl. Acids Res. 1992, 20, 4831-4837.
Means, G. E. and Feeney, R. E., Bioconjugate Chem. 1990, 1, 2-12 and references cited therein.
Partis, M. D., Griffiths, D. C., Roberts, G. C. and Beechley, R. D. J. Protein Chem. 1983, 2, 263-277.
Pope, N. M., Kulcinski, D. L., Hardwick, A., and Chang, Y., Bioconjugate Chem., 1993, 4, 166-171.
Reyes, R. A., and Cockerell, G. L., Nucl. Acids Res., 1993, 21, 5532-5533.
Ruth, J. L., Oligonucleotide-Enzyme Conjugates, in “Protocols for Oligonucleotide Conjugates”, Agrawal, S., Ed., 1994, p167-186.
Roychoudhury, R and Wu. R., Methods Enzymol. 1980, 65, 43-62.
Sambrook, J., Fritch, E. F., and Maniatis, T., Molecular Cloning: A Laboratory Manual. Cold Spring Harbor Laboratory Press, 1989, 1, p556.
Whitaker, J. E., Haugland, R. P., Ryan, D., Hewitt, P. C., Haugland, R. P. and Prendergrast, F. G., Anal. Biochem., 1992, 207, 267-279.

Zhu, Z., J. Chao, H. Yu and A. S. Waggoner (1994) Nucl. Acids. Res. 22: 3418-3422.

TABLE 1
Materials Appropriate for Attachment to Nucleic Acid Modified with
Nucleotides According to the Invention.
For use with SAc-Modified Nucleotides:

| | |
|---|---|
| Maleimide derivatized polystyrene tubes | Nunc, Naperville, IL |
| (Covalink ™ + SMCC) | |
| Acrylamide beads (bromoacetyl BioGel) | BioRad, Richmond, CA |
| Trisacryl GF2000 | IBF Corp; Fahy, et al., 1993. |
| Tresyl and epoxy resins | Toso Haas, |
| (Toyopearl ®) | Montgomeryville. PA |
| Organomercurial agarose beads | BioRad, Richmond, CA |
| (Affi-gel ® 501) | |
| Thiol-reactive fluorophores (e.g., I- | Molecular Probes, Eugene, OR |
| EDANS, Fluorescein-maleimide) | |
| Bromoacetyl cellulose beads | Sigma, St. Louis, MO |

### Appendix 1

APPENDIX 1.
Chemical/Biochemical Characterization of Functionalized Nucleotides

| | Chemical | tested with | DNA probe made |
|---|---|---|---|
| Nucleotide: | characterization | polymerases: | therewith? |
| Fluorescein- | UV/HPLC/ms¹ | TdT, T7, KF¹, e(−)KF | Yes |
| 12-dUTP | | PfU, (e−)PfU, Taq | |
| | | Taq-Stoffel | |
| PDP-dUTP | UV/HPLC | TdT, Taq, T7, KF | Yes |
| MCC-dUTP | UV/HPLC | TdT, Taq, T7, KF | Yes |
| SAc-dUTP | UV/HPLC | TdT, Taq, T7, KF | No |
| SAc-UTP | UV/HPLC | T3 RNA poly | Yes |

¹KF = Klenow Fragment of E. coli Polymerase 1. The (e−) notation refers to polymerases lacking the 3′,5′-exonuclease domain.

1. A nucleotide comprising the structure:
Phosphate-sugar-nucleobase-linker-F;
wherein F is a functional group selected from:

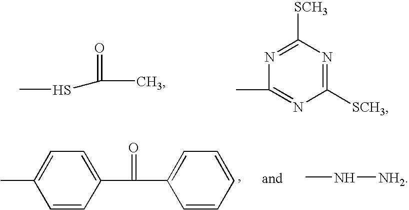

2. The nucleotide of claim 1 wherein said Linker is attached to said Nucleobase at the N-4 or C-5 position of said nucleobase when said nucleobase is a pyrimidine, or at the N-6, C-8 or C(N)-7 position of said nucleobase when said nucleobase is a purine.

3. The nucleotide of claim 1 wherein said nucleobase is selected from the group consisting of: adenine, cytosine, guanine, thymine, uracil and hypoxanthine.

4. The nucleotide of claim 1 wherein said linker is selected from the group consisting of:
—CH₂—(CH₂—CH₂)ᵥ—CH₂—NHC(O)-Q-; —CH₂—(CH₂—CH₂)_{V}—CH₂—C(O)—NH—C(O)-Q-;
—S—CH₂C(O)-Q-; —S—CH₂CH₂NH—C(O)-Q-; —O—CH₂C(O)-Q; —O—CH₂CH₂NH—C(O)-Q-;
—NH—(CH₂)ᵥ—NH—C(O)-Q-; 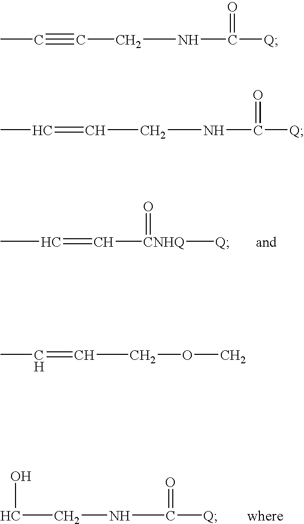
v=0,1,2,3, Q=—NH(CH₂)₆NH—, —NH—(CH₂)₂—NH, —(CH₂)₅NH—, —(CH₂)₂—C(O)—NH—(CH₂)₃—O—(CH₂)₂—O—(CH₂)₂—O—(CH₂)₃—NH—, —NH—[(CH₂)₂—O—)_{w}—(CH₂)₂—NH—, —(CH₂)₂ C(O)—NH—[(CH₂)₂—O]_{W}—NH—, and w=2,3,4,5.

5. The nucleotide of claim 1 wherein said nucleotide is selected from the group consisting of ATP, dATP, ddATP, GTP, dGTP, ddGTP, CTP, dCTP, ddCTP, UTP, dUTP, TTP and ddTTP.

6. The nucleotide of claim 1 wherein said phosphate moiety is a mono-, di-, tri-, or tetraphosphate group.

7. The nucleotide of claim 1 wherein said sugar moiety is a cyclic pyranofuranose sugar.

8. The nucleotide of claim 7 wherein said cyclic pyranofuranose sugar is selected from the group consisting of ribofuranosyl, 2′-deoxyribofuranosyl, and 2′,3′-dideoxyribofuranosyl.

9. The nucleotide of claim 1 wherein said sugar moiety is a cyclic non-furanose sugar.

10. The nucleotide of claim 9 wherein said cyclic non-furanose sugar is selected from the group consisting of oxetan, pyran or oxadiazepine.

11. The nucleotide of claim 1 wherein said sugar moiety is an acyclic sugar analog.

12. The nucleotide of claim 11 wherein said acyclic sugar analog is selected from the group consisting of phosphonomethoxyethyl, 2-oxyethoxymethyl, 2-hydroxymethoxymethyl, and 3-pentenyl.

13. A method of labeling a nucleotide of claim 1, said method comprising contacting said nucleotide with a detectable moiety comprising a reactive thiol group.

14. The method of claim 13 wherein said detectable moiety comprises a chromogenic dye, a fluorescent dye, a polypeptide or an enzyme.

15. A nucleotide labeled according to the method of claim 13.

16. A method of labeling a nucleic acid, said method comprising contacting said nucleic acid with a nucleotide of claim 15.

17. The method of claim 16 wherein said contacting is performed in the presence of a nucleic acid polymerase.

18. A method of labeling a nucleic acid, said method comprising contacting said nucleic acid with a nucleotide of claim 1.

19. The method of claim 18 wherein said contacting is performed in the presence of a nucleic acid polymerase.

20. The method of claim 18 further comprising contacting said nucleotide with a thiol-containing detectable moiety.

21. The method of claim 20, wherein said thiol-containing detectable moiety is a chromogenic moiety, a fluorescent dye, a polypeptide or an enzyme.

22. A polynucleotide comprising a nucleotide of claim 1.

23. A method of attaching a nucleic acid to a surface, said method comprising:
a) contacting said nucleic acid with a nucleotide of claim 1 in the presence of a nucleic acid polymerase, wherein said contacting results in the incorporation of said nucleotide into said nucleic acid or its complement;
b) contacting the nucleic acid of step (a) with a surface comprising a reactive group complementary to the functional group F on said nucleotide, wherein said contacting results in covalent attachment of said nucleic acid of step (a) to said surface.

24. The method of claim 23 wherein said surface is a plate, tube, bead or column matrix.

25. A kit comprising a nucleotide of claim 1.

26. The kit of claim 25, further comprising a nucleic acid polymerase, and packaging materials therefor.

27. A nucleotide comprising the structure:
Phosphate-Sugar-Nucleobase-F
wherein F is a functional group selected from: 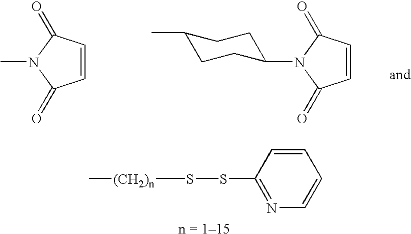
wherein said sugar is an acyclic sugar analog.

28. The nucleotide of claim 27 wherein said acyclic sugar analog is selected from the group consisting of phosphonomethoxyethyl, 2-oxyethoxymethyl, 2-hydroxymethoxymethyl, and 3-pentenyl.

29. A polynucleotide comprising a nucleotide of claim 27.

30. A kit comprising a nucleotide of claim 27.

31. The kit of claim 30, further comprising a nucleic acid polymerase, and packaging materials therefor.


SEQUENCE LISTING (5 sequences)

SEQ ID NO: 1 - DNA, 12 bp, Artificial sequence
  Synthetic oligonucleotide used for primer extension assays testing the incorporation of modified nucleotides.
cctggtcgtcgg

SEQ ID NO: 2 - DNA, 18 bp, Artificial sequence
  misc_feature (1)..(18): Synthetic oligonucleotide used in primer extension assays to test the incorporation of modified nucleotides.
ataataccgacgaccagg

SEQ ID NO: 3 - DNA, 17 bp, Artificial sequence
  Synthetic oligonucleotide used in primer extension assays to test the incorporation of modified nucleotides.
cctggtcgtcggcgtac

SEQ ID NO: 4 - DNA, 63 bp, Artificial sequence
  Synthetic oligonucleotide used in primer extension assays to test the incorporation of modified nucleotides.
gcttaccagtcatcgggtccaagtgtatagacgcatgagagtgtaggtacgccgacgaccagg

SEQ ID NO: 5 - DNA, 22 bp, Artificial sequence
  Synthetic oligonucleotide used in primer extension assays to test the incorporation of modified nucleotides.
gctaatcatggtcatagctgtt